(19) United States
(12) Patent Application Publication
(10) Pub. No.: US 2013/0296678 A1
(43) Pub. Date: Nov. 7, 2013

(54) COMBINATION STRUCTURAL POROUS SURFACES FOR FUNCTIONAL ELECTRODE STIMULATION AND SENSING

(71) Applicant: Cardiac Pacemakers, Inc., St. Paul, MN (US)

(72) Inventors: Steven R. Larsen, Lino Lakes, MN (US); Arthur J. Foster, Blaine, MN (US); Rakesj Radhakrishnan, Maple Grove, MN (US); Eric M. Petersen, Maple Grove, MN (US)

(21) Appl. No.: 13/868,294

(22) Filed: Apr. 23, 2013

Related U.S. Application Data
(60) Provisional application No. 61/637,555, filed on Apr. 24, 2012.

(51) Int. Cl. A61N 1/05; A61B 5/04

(52) U.S. Cl. CPC A61N 1/05; A61B 5/04
USPC 600/373; 607/116; 29/825

(57) ABSTRACT

An implantable medical lead including a proximal end portion and a distal end portion and an electrical conductor electrically connected to the proximal end portion of the lead body. Also, the lead has at least one electrode connected to the distal end portion of the lead body and connected to the electrical conductor. The electrode includes a conductive base structure, a first set of pores formed on an outer surface of the conductive base structure, the first set of pores having an average first pore dimension of between about ¼^{th} and about 1/100^{th} an electrode dimension, and a second set of pores formed on at least a portion of the first set of pores, the second set of pores having an average second pore dimension of between about ¼^{th} and about 1/100^{th} average first pore dimension.

### CROSS-REFERENCE TO RELATED APPLICATION

This application claims priority to U.S. Provisional Application No. 61/637,555, filed Apr. 24, 2012, which is herein incorporated by reference in its entirety.

### TECHNICAL FIELD

The present invention relates to medical devices. More specifically, the invention relates to an electrode and an implantable medical lead having the electrode for providing stimulation or for sensing.

### BACKGROUND

Implantable medical devices, such as electrical stimulators or sensors, are used in different therapeutic or medical applications. In some implantable medical devices, the electrical stimulator or sensor delivers electrical pulses to a target tissue site within a patient with the aid of one or more medical leads. The medical leads are coupled to the implantable medical device at one end while the other end carrying electrodes is placed at the target tissue site. The electrodes are used for stimulating body tissues or in sensing applications.

The ability of an electrode to transfer current is proportional to the surface area of the electrode. An important requirement in the design of the medical leads is smaller diameter that can be gained by reduced thresholds with higher impedance. As the diameter decreases, current density and tissue interface impedance increases, however sensing ability decreases which may be undesirable. Thus, there is a need for a medical lead and an electrode with the medical lead that is capable of increased sensing or stimulation event as the diameter of the lead decreases.

### SUMMARY

Example 1 is an implantable medical lead used for stimulating or sensing a target tissue. The implantable medical lead has a lead body, including a proximal end portion and a distal end portion and an electrical conductor electrically connected to the proximal end portion of the lead body. Also, the lead has at least one electrode connected to the distal end portion of the lead body and connected to the electrical conductor. The electrode includes a conductive base structure, a first set of pores formed on an outer surface of the conductive base structure, the first set of pores having an average first pore dimension of between about ¼^{th} and about 1/100^{th} an electrode dimension, and a second set of pores formed on at least a portion of the first set of pores, the second set of pores having an average second pore dimension of between about ¼^{th} and about 1/100^{th} the average first pore dimension.

Example 2 is the implantable medical lead of Example 1, wherein the conductive base structure is made of platinum or platinum alloy.

Example 3 is the implantable medical lead of Examples 1, wherein the conductive base structure is made of stainless steel, nitinol, nickel-cobalt alloy, titanium, gold, niobium, tantalum, ruthenium, palladium, or palladium alloy.

Example 4 is the implantable medical lead of any of Examples 1-3, wherein the conductive base structure has a mushroom, helical, cylindrical, ribbon, or a spherical shape.

Example 5 is the implantable medical lead of any of Examples 1-4, wherein the electrode dimension is one of the length, width, diameter, or thickness of the electrode.

Example 6 is the implantable medical lead of any of Examples 1-5, wherein the average first pore dimension is between about 10 and about 1000 μm.

Example 7 is the implantable medical lead of any of Examples 1-6, further comprising the first set of pores having an average first pore dimension of between about 1/15^{th} and about 1/16^{th} an electrode dimension.

Example 8 is the implantable medical lead of any of Examples 1-7, further comprising the second set of pores having an average second pore dimension of between about 1/15^{th} and about 1/16^{th} average first pore dimension.

Example 9 is the implantable medical lead of any of Examples 1-8, further comprising a third set of pores formed on at least a portion of the second set of pores, the third set of pores having an average third pore dimension of between about ¼^{th} and about 1/100^{th} average second pore dimension.

Example 10 is the implantable medical lead of any of Examples 1-9, further comprising the third set of pores having an average third pore dimension of between about 1/15^{th} and about 1/16^{th} average second pore dimension.

Example 11 is a method for manufacturing an electrode that is included with an implantable medical lead body used for simulating or sensing a target tissue. The method includes creating an electrode with a porous structure by forming a conductive base structure. The method further includes forming a first set of pores on at least a portion of the conductive base structure, the first set of pores having an average first pore dimension of between about ¼^{th} and about 1/100^{th} an electrode dimension. The method further includes forming a second set of pores having a plurality of pores having an average second pore dimension of between about ¼^{th} and about 1/100^{th} the average first pore dimension.

Example 12 is the method of Example 11, wherein one or both of the forming steps are performed using a laser ablation process.

Example 13 is the method of Examples 11 and 12, wherein one or both of the forming steps are performed using a laser ablation process.

Example 14 is the method of any of Examples 11-13, wherein one or both of the forming steps are performed using one of a chemical etching, micro-abrasive blasting, and an electrical discharge machining (EDM) melt process.

Example 15 is the method of any of Examples 11-14, one or both of the forming steps are performed using one of a material deposition process and dealloying process.

Example 16 is the method of any of Examples 11-15, further comprising forming a third set of pores having a plurality of pores having an average third pore dimension of between about ¼^{th} and about 1/100^{th} the average second pore dimension

Example 17 is the method of any of Examples 11-16, further comprising forming a fourth set of pores having a plurality of pores having an average fourth pore dimension of between about ¼^{th} and about 1/100^{th} the average third pore dimension.

Example 18 is the method of any of Examples 11-17, wherein the fourth set of pores is formed by a process that adds to, removes, displaces, and/or changes the material of the conductive base structure.

Example 19 is the method of any of Examples 11-18, wherein the forming steps are performed using a laser ablation process.

Example 20 is the method of any of Examples 11-19, wherein the forming steps are performed using one of a chemical etching, micro-abrasive blasting, and an EDM melt process.

Example 21 is the method of any of Examples 11-20, wherein the forming steps are performed using one of a material deposition process and de-alloying process.

### BRIEF DESCRIPTION OF THE DRAWINGS

FIG. 1A is a schematic view of an implantable medical device in a cardiac rhythm management (CRM) system, according to various embodiments.

FIG. 1B is a schematic view of an implantable medical device in a neurostimulation system, according to various embodiments.

FIG. 2 is a schematic view of a medical electrical lead in accordance with some embodiments.

FIGS. 3A, 3B, 3C, 3D, and 3E illustrate exemplary shapes of a medical electrode, in accordance with some embodiments.

FIGS. 4A and 4B are perspective and sectional views of a medical electrode having a porous surface.

FIGS. 5A and 5B are schematic views of an electrode surface with a porous structure, in accordance with some embodiments.

FIGS. 6A, 6B, and 6C are schematic views of a porous structure with two pore sets, in accordance with some embodiments.

FIGS. 7A, 7B, 7C, and 7D are schematic views of porous structures with a varying number of pore sets, in accordance with some embodiments.

FIG. 8 is a flowchart of a method for creating a porous structure, in accordance with some embodiments.

FIG. 9 is a flowchart of a method for creating a porous structure, in accordance with some embodiments.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

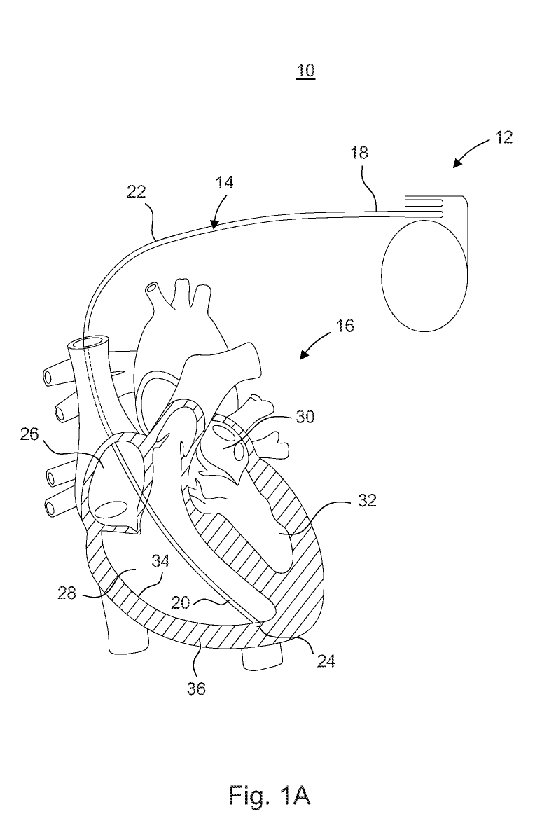

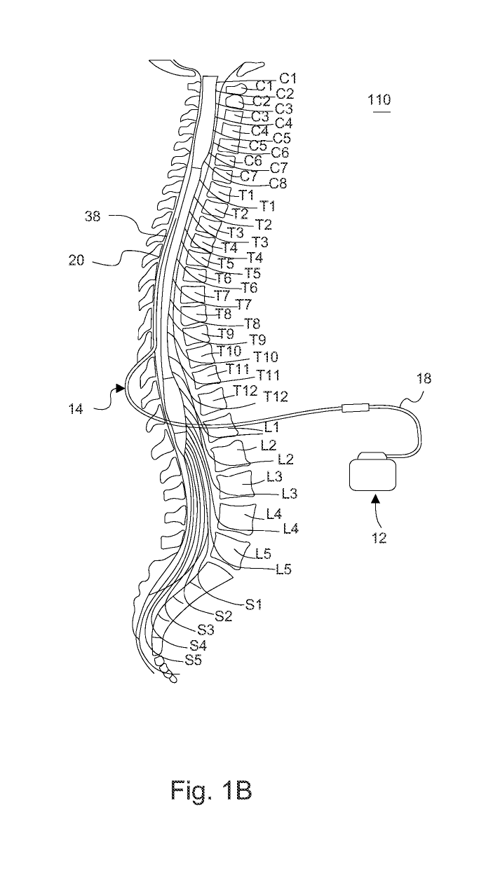

FIGS. 1A and 1B show exemplary medical applications for using an implantable lead device. In particular, these figures show different anatomical locations within the body wherein an implantable lead can be utilized.

FIG. 1A is a schematic view of an implantable cardiac rhythm management (CRM) system 10. As shown, the system 10 includes an implantable pulse generator (IPG) 12 and an implantable lead 14, which extends from a proximal end portion 18 to a distal end portion 20. As shown in FIG. 1, the heart 16 includes a right atrium 26, a right ventricle 28, a left atrium 30 and a left ventricle 32. It can be seen that the heart 16 includes an endocardium 34 covering the myocardium 36. In some embodiments, as illustrated, a fixation helix 24, located at the distal end portion 20 of the lead 14, penetrates through the endocardium 34 and is embedded within the myocardium 36. In some embodiments, the fixation helix 24 is electrically active and thus operates as a helical electrode 38 for sensing the electrical activity of the heart 16 and/or applying a stimulating pulse to the right ventricle 28. In some embodiments, the CRM system 10 includes a plurality of leads 14. For example, it may include a first lead 14 adapted to convey electrical signals between the pulse generator 12 and the right ventricle 28 and a second lead (not shown) adapted to convey electrical signals between the pulse generator 12 and the right atrium 26 or coronary veins (not shown).

FIG. 1B is a schematic view of a representative implantable neurostimulation (e.g., spinal cord stimulation) system 110. As shown in FIG. 1B, the neurostimulation system 110 includes an IPG 112, which generates electrical stimulation pulses, and a lead 14 extending from the pulse generator 112 to a desired stimulation site. The lead portion 14 has a proximal end portion 18 and a distal end portion 20 and includes an electrode 38 or plurality of electrodes 38 at or near the distal end portion 20. As further shown in FIG. 1B, C1-C8 are the cervical vertebrae and nerves, T1-T12 are the thoracic vertebrae and nerves, L1-L5 are the lumbar vertebrae and nerves, and S1-S5 are the sacrum and coccyx and the sacral nerves. Other implantable neurostimulation systems include deep brain stimulation and peripheral (e.g., vagal) nerve stimulation systems.

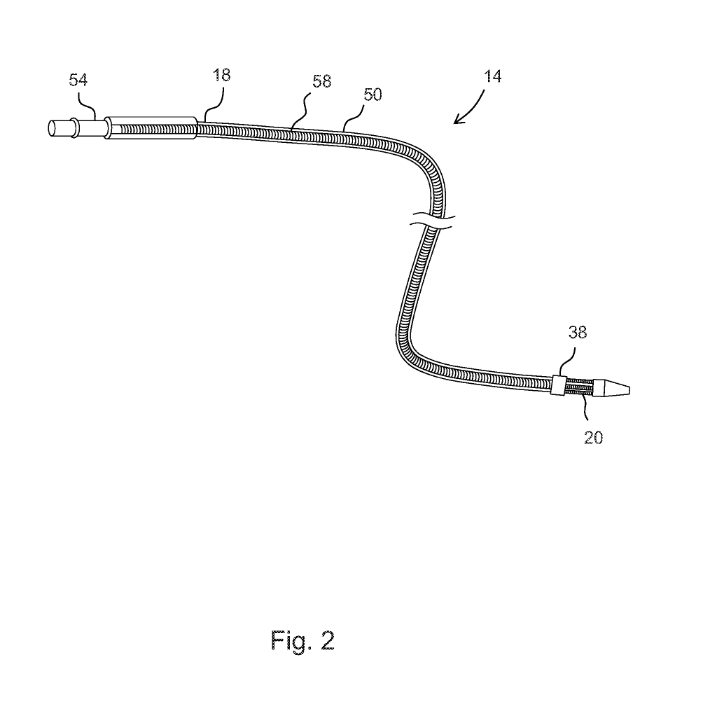

FIG. 2 is a schematic view of a medical electrical lead 14. The lead 14 is adapted to deliver electrical pulses to stimulate a heart 16 or nervous system and/or to receive electrical pulses to monitor the heart 16 or nervous system. According to some embodiments, the lead 14 can be sized and configured to be delivered near the vagus nerve, the peripheral nerves, the spinal cord, or the heart 16. The medical electrical lead 14 includes an elongated lead body 50 having opposed proximal and distal ends 18 and 20. The lead body 50 is formed from a bio-compatible insulative material, for example, silicone rubber, polyurethane, or the like. A connector 54 is operatively associated with the proximal end portion 18 of the lead body 50. The connector 54 may be of a standard type, size or configuration. Connector 54 is electrically connected to the electrode 38 by way of a conductor coil 58 that extends through the interior lumen of lead body 50. Conductor coil 58 is generally helical in configuration and includes one or more conductive wires or filaments. At least one electrode 38 is operatively associated with the distal end portion 20 of the lead body 50. The electrode 38 can be formed from one or more conductive materials. Examples of conductive materials include, but are not limited to, platinum, stainless steel, nitinol, MP35N, titanium, a platinum-iridium alloy, and combinations thereof. In some embodiments, the electrode 38 is disposed proximal to the distal end portion 20 of the lead 14. Alternatively, the electrode 38 can be located along the lead body 50 between the proximal end portion 18 and the distal end portion 20. According to yet another embodiment, the electrode 38 can be a tip electrode. A tip electrode is located at the very distal end portion 20 of the lead body 50 and is commonly employed in left ventricular leads. Multiple electrodes 38 may also be utilized according to some embodiments.

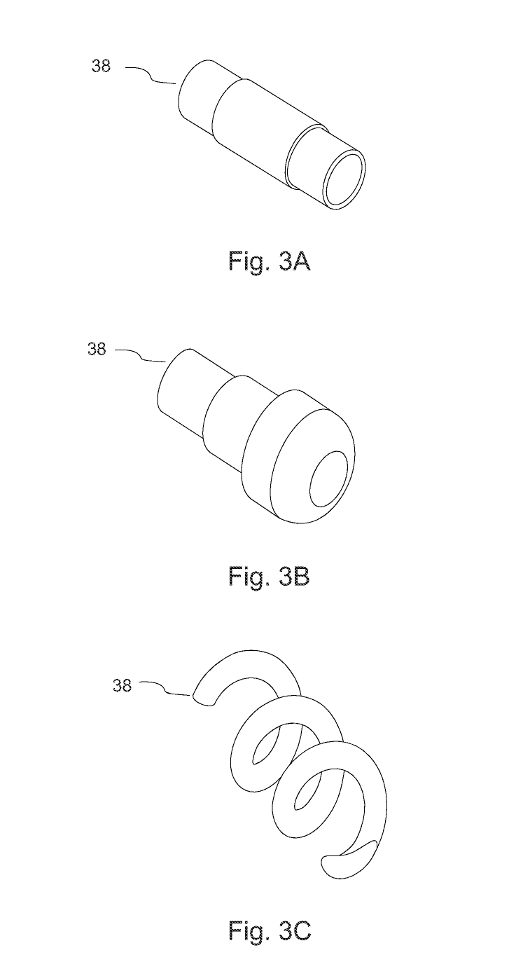

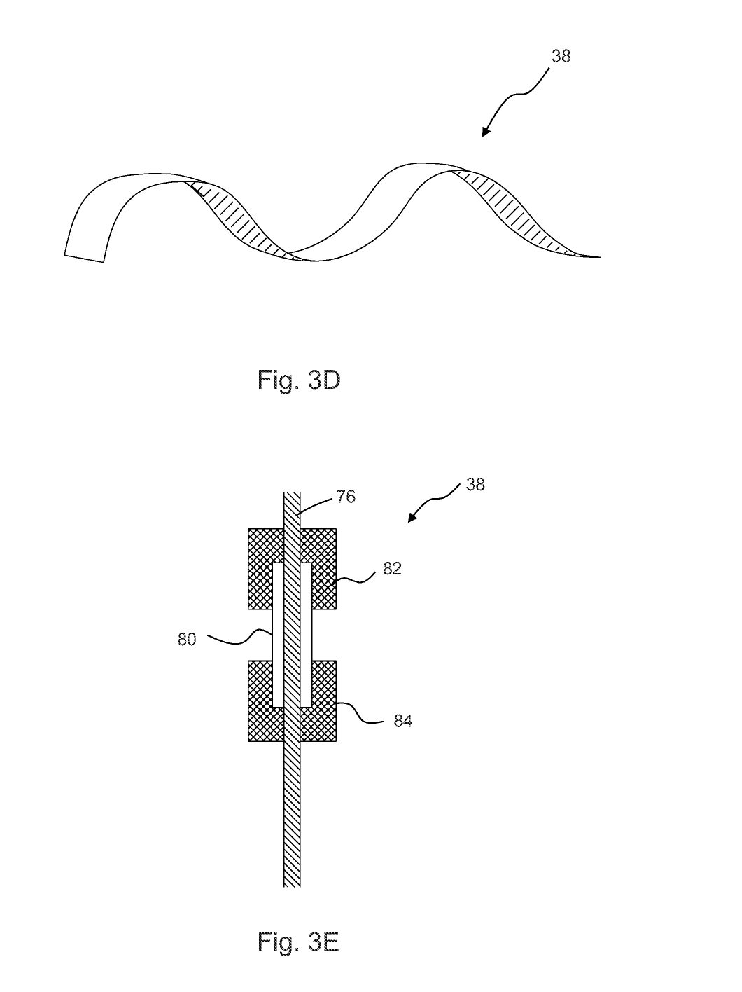

FIGS. 3A-3E show embodiments of the medical electrode 38 used for stimulating body tissue by delivering stimulation energy to a desired site. FIG. 3A shows a ring electrode 38 in the form of a cylindrical shape. In some embodiments, the electrode 38 can be a mushroom shape that includes an umbrella-like cap-and-stem form. In other embodiments, the electrode 38 can be a helically shaped rod or a flexible ribbon as illustrated in FIGS. 3C and 3D. FIG. 3E shows an electrode 38 in the form of a system having a positively-charged stent 82, a negatively-charged stent 84, and a tubular dielectric material 80 along the vagus nerve 76. The tubular dielectric insulator 80, connects to a portion of the positively-charged stent 82 on one end and connects to a portion of the negatively-charged stent 84 on the other end. Examples of dielectric insulator materials include, but are not limited to, polymers, ionic crystals, glass, and ceramics.

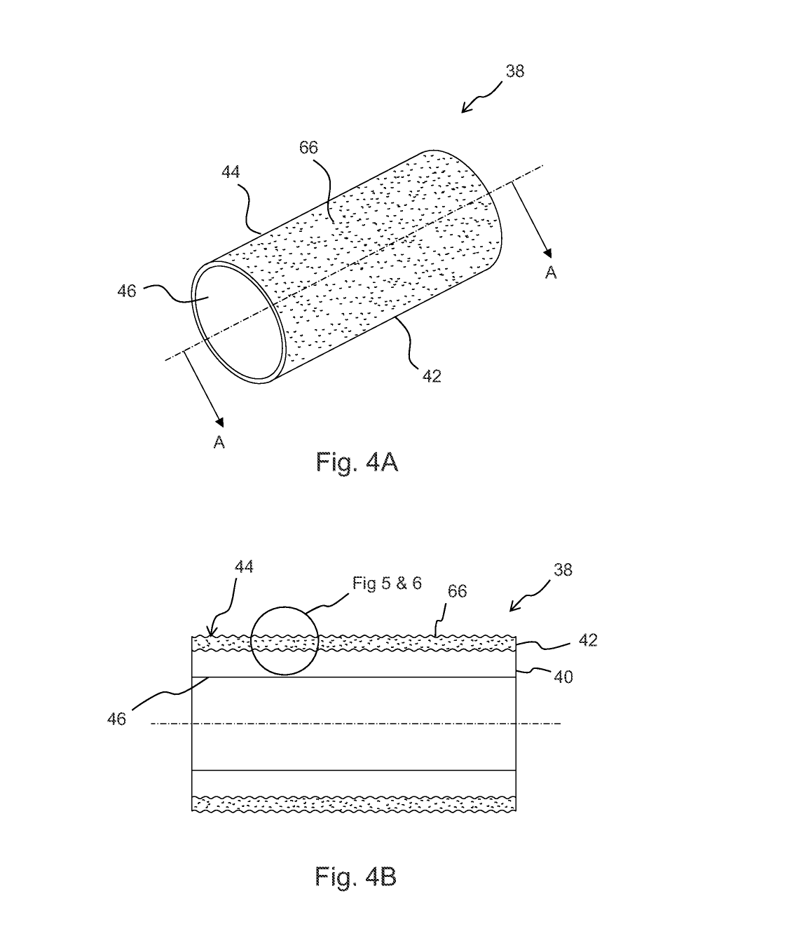

FIGS. 4A and 4B are perspective and sectional views of an electrode 38 having a porous surface 66. FIGS. 4A and 4B show an electrode 38, including a porous surface 66 for increasing conductivity of the electrical stimulations delivered by the electrode 38 and for improving sensing of electrical activity by the electrode 38. As shown in FIG. 4A, a porous surface 66 is located along the outer surface 44 of the electrode 38. As shown in FIG. 4B, the electrode 38 comprises a conductive base structure 40 and a porous structure 42. The conductive base structure 40 is coupled to the porous structure 42 that creates a porous surface 66 on the electrode 38. The conductive base structure 40 is formed into the desired macroscopic shape of the electrode 38 and may provide material for constructing the porous structure 42. According to various embodiments, the conductive base structure 40 may comprise one or more conductive materials. Examples of conductive materials include, but are not limited to, stainless steel, nitinol, platinum, palladium, titanium, niobium, tantalum, ruthenium, rhodium, or gold, or an alloy of two or more metals, for example, nickel-cobalt alloy, and combinations thereof

As shown in FIG. 4B, the porous structure 42 is coupled to a surface of the conductive base structure 40 portion of the electrode 38. In various embodiments, the porous structure 42 is coupled to at least a portion of the base structure 40 of the electrode 38. In some of these embodiments, the porous structure 42 is present on the outer diameter or outer surfaces 44 of the electrode 38. In other embodiments, the porous structure 42 may be present on the inner diameter or internal surfaces 46 of the electrode 38. The porous structure 42 on the surface of the electrode 38 may be described as smooth or rough, soft or hard, coarse or fine. The porous structure 42 increases the surface area of the electrode 38 to enable a low sensing impedance and improved sensing capability in a cardiac rhythm management (CRM) system or a neurostimulation system.

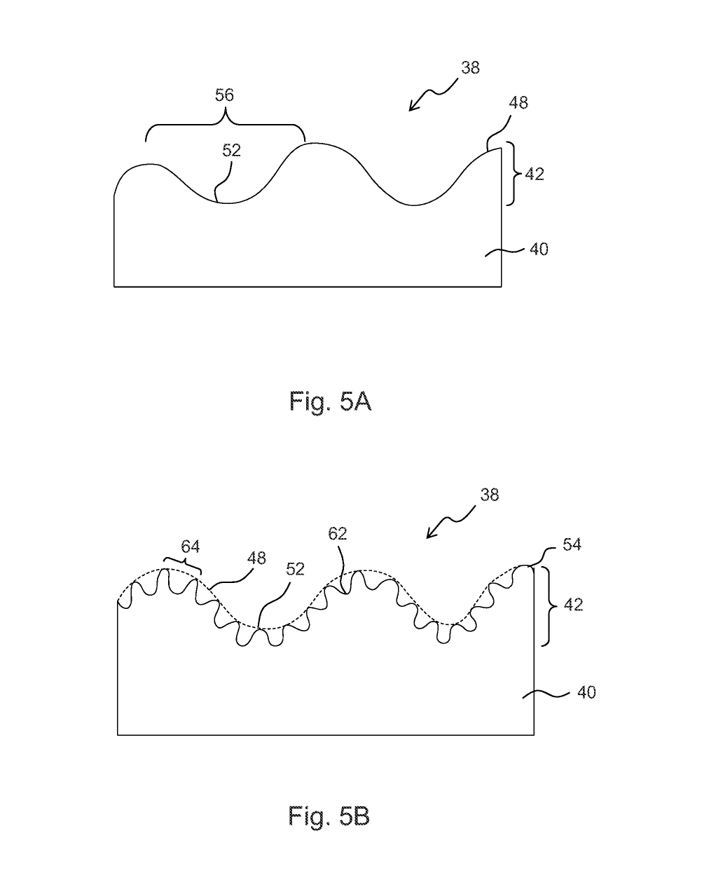

FIGS. 5A and 5B are schematic views of an electrode surface with a porous structure 42, in accordance with some embodiments. As shown in FIG. 5A, the electrode surface has a porous structure 42 with a single set of pores 52, a first set 48, coupled to the surface of the conductive base structure 40. A first set 48 of pores 52 can be created by adding material to, removing material from, or by modifying or shifting the material of the conductive base structure 40. In various embodiments, a first pore set 48 can be created by removing material from the base structure 40 by, for example, laser ablation processing, EDM melt processing, sintering, or chemical etching. In some embodiments, the first pore set 48 can be created by adding material to the conductive base structure 40, for example, plating or depositing a material in a powdered or fragmented form. In other embodiments, the first pore set 48 can be created by shifting the material of the conductive base structure 40, for example, by using micro-abrasive blasting.

In various embodiments, the porous structure 42 has at least two pore sets. As shown in FIG. 5B, the porous structure 42 includes the first set 48 of pores 52 and a second set 54 of pores 62. The first set 48 of pores 52 is coupled to the surface of the conductive base structure 40. The second set 54 of pores 62 is coupled to the surface of the first set 48 of pores 52. The pores 52 of the first set 48 are substantially smaller than the pores 62 of the second set 54. By coupling the second pore set 54 to the first pore set 48, the porous structure 42 may couple a set of micro-porous pores to the surface with a set of macro-porous pores. An electrode 38 with a porous structure 42 having multiple sets of pores 48, 54 as described herein significantly increases the effective surface area and activity of the electrode 38.

A “seed process” can be the initial process used to create the first set 48 of pores 52 with a corresponding first pore dimension 56 on the conductive base structure 40. In various embodiments, the seed process is selected based on a particular dimension of the electrode 38, for example, the diameter, length, width, or thickness of the electrode 38. Electrode dimensions can significantly vary depending on the form of the electrode 38. For example, the dimension of a ring electrode 38 may range from about 0.5 millimeters (mm) to 4 mm in length, about 1 mm to 4 mm in diameter, and about 0.025 to 0.050 mm in thickness, while the dimension of a ribbon electrode dimension may range from about 5 mm to 15 mm in length, about 0.5 to 2 mm in width, and about 0.010 to 0.050 mm in thickness. In various embodiments, the seed process can create the first pore dimension 56 ranging from about ¼^{th} to 1/100^{th} of a particular dimension of an electrode 38. In some of these embodiments, the seed process used to create the first pore dimension 56 may be based on the smallest dimension of the electrode 38. For example, a ring electrode 38 with a 4 mm length and a 1 mm diameter may use a seed process that creates a set of pores with an average pore dimension of between about ¼^{th} to about 1/100^{th} the diameter dimension. The resulting set of pores will have a first pore dimension 56 ranging between about 10 micrometers (μm) and about 250 μm. In various embodiments, the pore dimension 64 of a second set 54 of a porous structure 42 is increased or decreased by ¼^{th} to 1/100^{th} of the dimension 56 of the pores 52 of the first set 48. The average pore dimension 56, 64 of each pore set 48, 54 of the porous structure 42 can be optimized for minimizing sensing impedance in order to detect intrinsic signals from tissue and/or to increase bonding integrity between the electrode 38 and the lead body 50.

In some embodiments, the aspect ratio of the average pore depth relative to the average pore dimension 56, 64 can be on the order of 1:1. In other embodiments, the aspect ratio of the average pore depth relative to the average pore dimension 56, 64 can range from approximately 1:4 to approximately 3:2. For example, an electrode 38 having a first set 48 of /pores 52 with an average pore dimension 56 of 100 μm and an aspect ratio of 1:4 will yield pores 52 with an average pore depth of 25 μm. In some embodiments, the average pore depth may also depend on a particular dimension of the electrode 38, for example, the diameter, length, width, or thickness of the electrode 38.

The surface area of a conductive base structure 40 can be significantly increased when a porous structure 42 is formed into and/or onto the conductive base structure 40. The surface area of the conductive base structure 40 can change depending on the number of pore sets and the pore-dimension ratio, which is the ratio of the pore dimension relative to the dimension of the electrode or the adjacent pore set. Table 1 below provides computational estimates of the predicted percentage increase of the surface area of a conductive base structure 40 with a porous structure 42 based on the pore-dimension ratio and the number of pore sets. The surface area increase data provides a comparison of the surface area of a conductive base structure 40 with a porous structure 42 relative to the surface area of a non-porous conductive base structure 40.

TABLE 1

| Pore- | No. of | Surface Area |
|---|---|---|
| Dimension | Pore | Increase |
| Ratio | Sets | (percent) |
| 1/7 | 4 | 3500 |
| 1/11 | 4 | 3800 |
| 1/15 | 3 | 5000 |
| 1/19 | 3 | 6000 |
| 1/23 | 3 | 3700 |
| 1/27 | 2 | 2100 |
| 1/31 | 2 | 2400 |
| 1/35 | 2 | 2500 |
| 1/39 | 2 | 2600 |

The data provided above was generated using an algorithm-based simulation that creates fractal surfaces in the form of five-sided pyramid-shaped structures. The simulation collects data on the increased surface area by iteratively creating smaller pyramid structures on the surface of each initial pyramid structure until a predetermined limit value relating to the feature dimension is reached. Based on the presented data, the porous structure 42 can increase the maximum surface area of an electrode 38 by approximately 2100 to 6000 percent when using a pore-dimension ratio ranging from 1/7th to 1/39th. For example, forming a porous structure 42 having three pore sets 48, 54, 58 with a pore-dimension ratio of between 1/15^{th} and 1/19^{th} would yield a conductive base structure 40 with an increased surface area ranging between 5000 percent and 6000 percent.

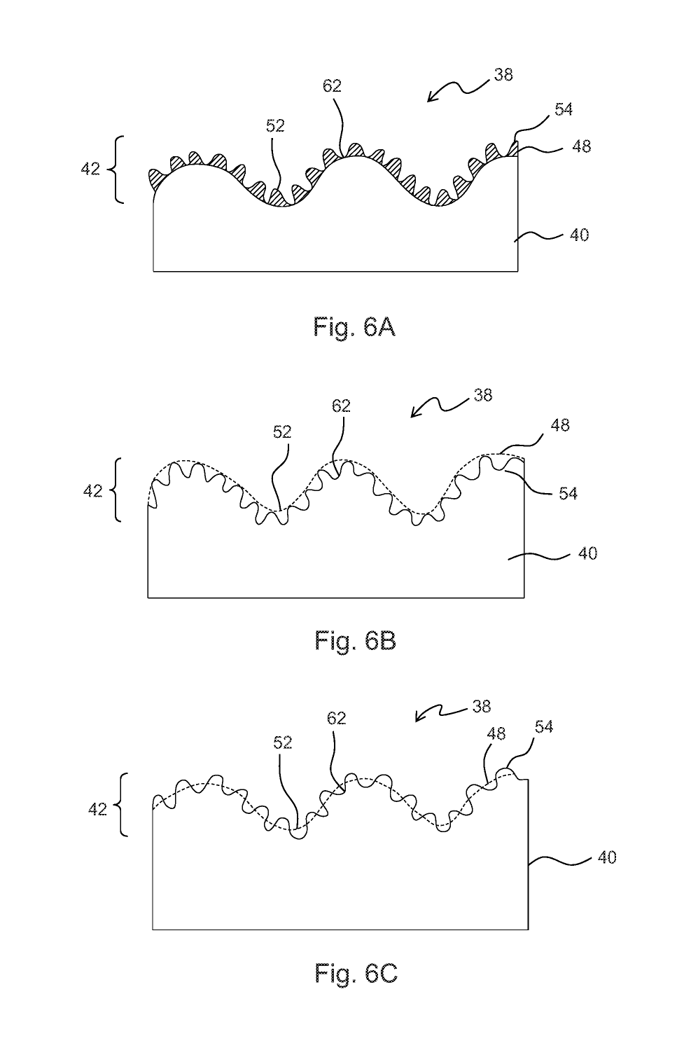

FIGS. 6A-6C are schematic views of a porous structure 42 with two pore sets 48, 54, in accordance with some embodiments. The figures illustrate the different ways a second pore set 54 can be created and coupled to the first pore set 48. The second set 54 of pores 62 can be created by adding material to, removing material from, or by modifying or shifting the material of the first pore set 48. In various embodiments, the pores 62 of a second set 54 can be created into and/or on top of the first pore set 48 using similar processes as those used for creating the first pore set 48, as discussed in previous sections. However, the second pore set 54 may, in some embodiments, use other types of processes since the pores 62 of the second set 54 are significantly smaller than those of the first set 48. As shown in FIG. 6A, a second pore set 54 can be created by adding material to the conductive base structure 40, for example, adding platinum black, iridium oxide (IrOx), titanium nitride (TiNi), or titanium carbide. Platinum black is a fine black powder of metallic platinum having good conductivity and porosity. Iridium oxide, titanium nitride, and titanium carbide are good conductive biocompatible materials. Materials may be added to the conductive base structure 40, for example, by sintering, plating, material deposition, chemical vapor deposition (CVD), physical vapor deposition sputtering (PVD), or atomic layer deposition (ALD) techniques.

In other embodiments, as shown in FIG. 6B, a second pore set 54 can be created by removing material from the base structure 40 by, for example, dealloying, electric discharge machining (EDM) melt processing, laser ablation processing, or chemical etching. In yet other embodiments, as shown in FIG. 6C, the second pore set 54 can be created by shifting the material of the conductive base structure 40, for example, by using micro-abrasive blasting or other similar processes.

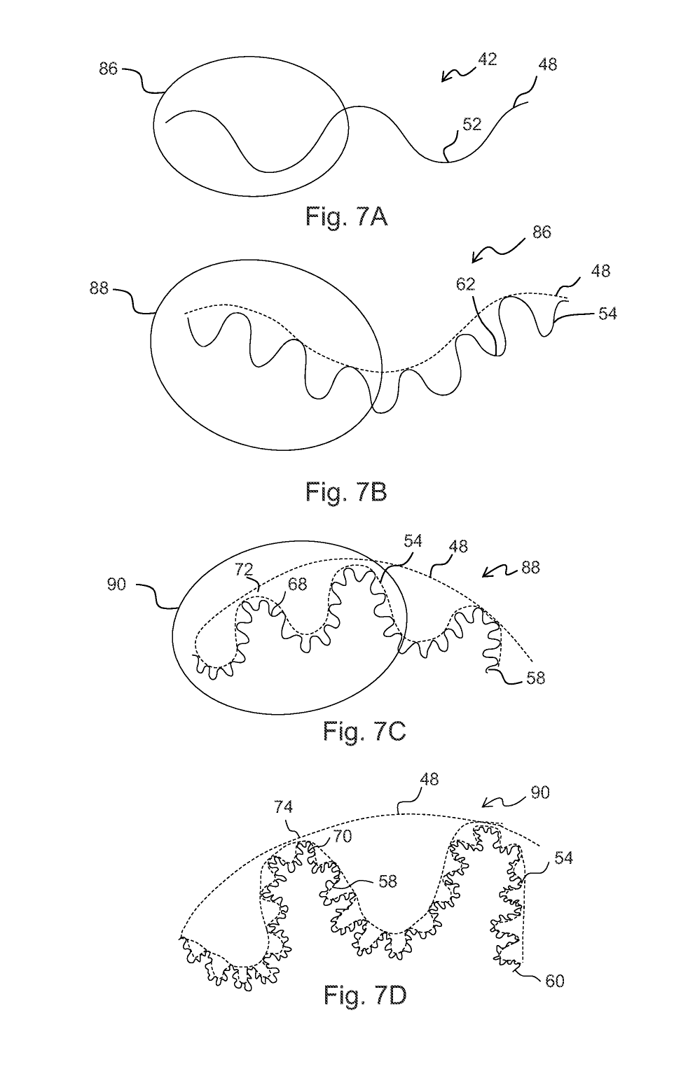

FIGS. 7A-7D are schematic views of porous structures 42 with a varying number of pore sets. As shown in FIG. 7A and 7B, a porous structure 42 may have a single set of pores or two sets of pores, as previously discussed. A single set of pores comprises a first set 48 of pores 52 and two sets of pores comprises a first and second set 48, 54 of pores 52, 62. In some embodiments, a third set 58 of pores 68 can be coupled to the surface of the second set 54 and a fourth set 60 of pores 70 can be coupled to the surface of the third pore set 58, as shown respectively in FIGS. 7C and 7D. In other embodiments, a similar method can be used to couple subsequent pore sets to the fourth pore set 60 that creates a porous structure 42 comprising five to ten sets of pores.

In various embodiments, the pore dimension 72, 74 of the third set 58 and the fourth pore set 60 is increased or decreased by ¼^{th} to 1/10^{th} of the pore dimension 64, 72 of the second and the third set 54, 58, respectively. Essentially, the pore dimension 56, 62, 72, 74 of each additional pore set 48, 54, 58, 60 of the porous structure 42 is increased or decreased by ¼^{th} to 1/100^{th} of the dimension of the pores 56, 62, 72 of the adjacent pore set 48, 54, 58 or the surface structure 50 that the additional pore set couples to. In some embodiments, the porous structure 42 can have three pore sets 48, 54, 58 with a pore-dimension ratio ranging between 1/15^{th} and 1/19^{th}, yielding a conductive base structure 40 with an estimated increased surface area of 5000 percent to 6000 percent. For this reason, in various embodiments, the range of the average pore dimension 56, 62, 72, 74 of the porous structure 42 comprising a plurality of pores sets 48, 54, 58, 60 may range from about 1 nanometer (nm) up to about 1000 μm. For example, using a 1/100^{th} pore-dimension ratio can yield a porous structure 42 with a pore dimension 56, 62, 72, 74 of 1000 μm at the innermost first pore set 48 and 1 nm at the outermost last pore set 60. If the first pore set 48 contains a pore dimension 56 of about 1000 μm, for example, then a second pore set 54 adjacent to the first pore set 48 may have a decreased pore dimension 64 of about 100 μm. Similarly, a third pore set 58 adjacent to the second pore set 54 may have a decreased pore dimension 72 dimension of about 10 μm, and so on.

A porous structure 42 with multiple pore sets can be formed using the processes discussed herein to add to, remove from, or modify the material that each pore set 48, 54, 58, 60 directly couples to. In various embodiments, a combination of pore set forming processes can be used to incrementally decrease the pore dimension 56, 62, 72, 74 of each added pore set 48, 54, 58, 60 in the porous structure 42. A combination of processes can include, for example, micro-abrasive blasting or chemical etching to create pores 52, 62, 68, 70 ranging in the 10-100 μm size range, followed by de-alloying that creates pores 52, 62, 68, 70 in the smaller 1-10 nm size range. In some embodiments, even smaller pores measuring less than one nanometer may be created by methods such as chemical vapor deposition (CVD), physical vapor deposition sputtering (PVD), or atomic layer deposition (ALD) techniques. Using a combination of processes can create a porous structure 42 in which each pore set 48, 54, 58, 60 added to the porous structure 42 can substantially increases the overall conductivity of the electrode 38. In some embodiments, because the dimension 56, 62, 72, 74 of the pores 52, 62, 68, 70 decreases as they approach the surface of the electrode 38, the geometric structure of the porous structure 42 becomes increasingly finer toward its surface with each added set 48, 54, 58, 60 of pores 52, 62, 68, 70.

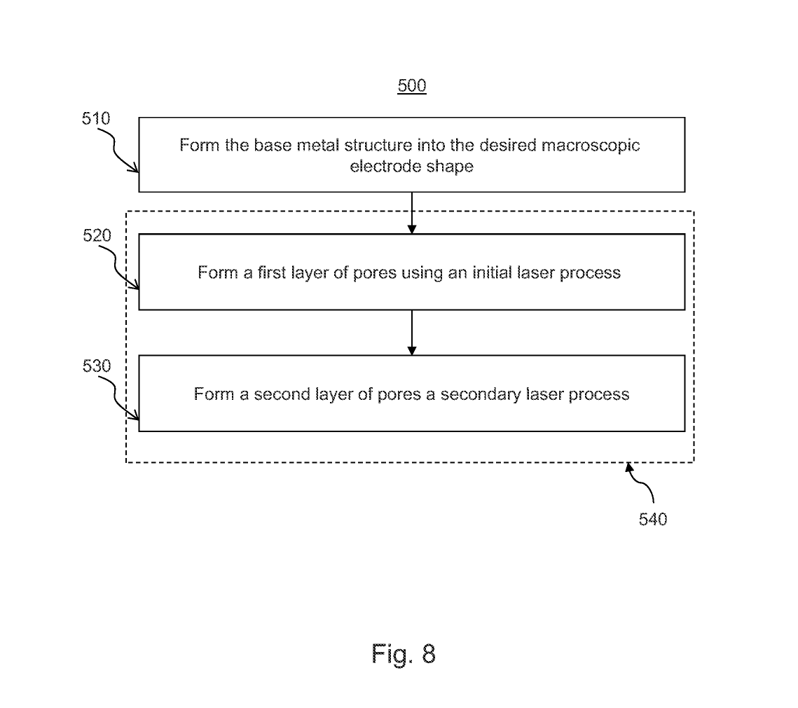

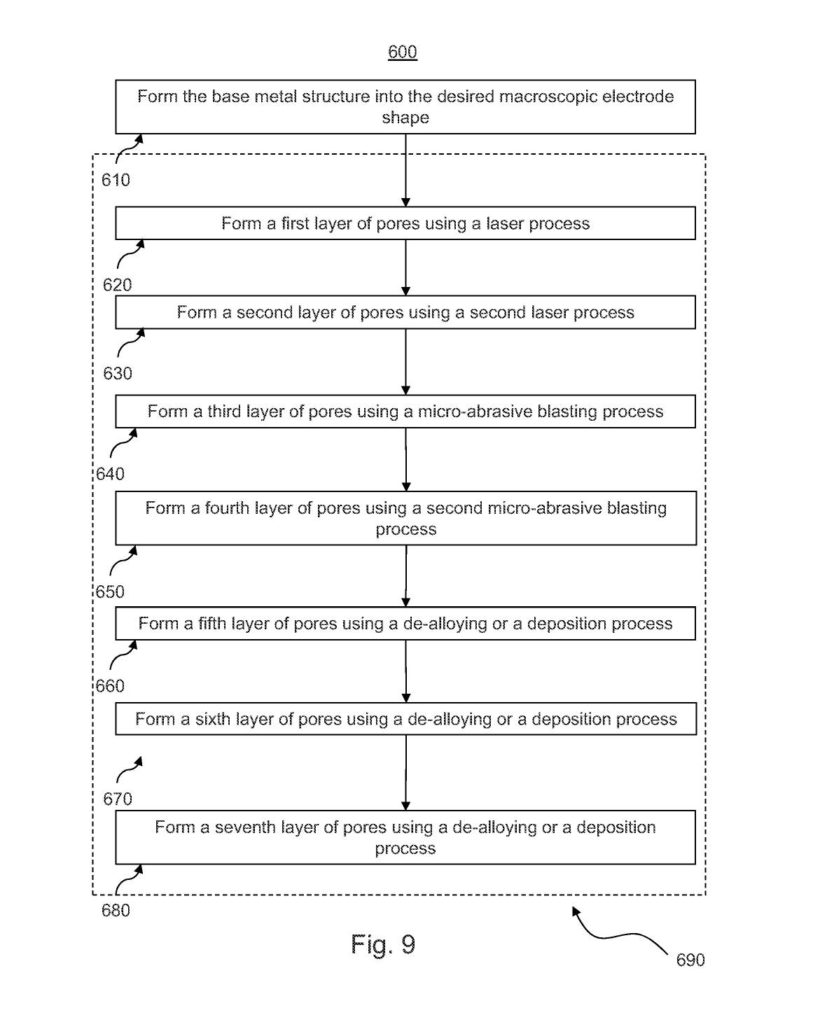

FIGS. 8 and 9 are flow charts illustrating a method 500, 600 of manufacturing an electrode 38 having a conductive base structure 40 and a porous structure 42 with various numbers of pore sets 48, 54, 58, 60. The method 500, 600 may be used with a metallic electrode 38 included with an implantable medical lead 14 in a number of various applications including, for example, a CRM system or a neurostimulation system. The method is useful in allowing a manufacturer to increase the surface area of an electrode 38 that contacts a desired tissue site by providing higher functional electrode stimulation and sensing capability.

The method 500 includes creating a porous structure 42 with two porous pore sets 48, 54. In various embodiments, the pores 52, 62 of each added set 48, 54 of pores 52, 62 are incrementally decreased by 1/100^{th} and each pore set 48, 54 is formed using a separate process. The method 500 includes creating a conductive base structure 40 by using a metallic material to form a basic electrode shape with an overall length of 1000 μm (block 510). Possible options for basic electrode shapes and the composition of the base material structure 40 are discussed herein.

Once the conductive base structure 40 is formed, a first set 48 of pores 52 can be formed on at least a portion of the surface of the conductive base structure 40 (block 520). In some embodiments, the first pore set 48 is formed by creating pores 1/100^{th} the dimension of the overall length of the electrode 38 by using a laser ablation process, yielding an average pore dimension 56 of 100 μm. The laser ablation process is a process that creates pores by removing material from the conductive base structure 40. In other embodiments, alternative processes may also be used to remove material to create the pores 52 into the first pore set 48, for example, EDM melt processing, sintering, or chemical etching. In some embodiments, a process that adds material to the conductive base structure 40 may be used to create pores 52 into the first pore set 48, for example, plating or depositing a material in a powdered or fragmented form.

After forming the first pore set 48, a second pore set 54 with pores 62 of a smaller dimension 64 is formed on at least a portion of the surface of the first pore set 48 (block 530). The second pore set 54 can be formed by creating pores 1/100^{th} the dimension of the pores 56 of the first pore set 48 by using a second laser ablation process, yielding a second pore set 54 with an average pore dimension 64 of about 10 μm.

The first and second pore sets 48, 54 together create the porous structure 42 on the surface of the electrode 38 (block 540).

The method 600 includes creating an embodiment of an electrode 38 having a porous structure 42 with seven porous pore sets. The pore dimension of each pore set decreases to 1/100^{th} the dimension of the pores of the previous pore set and are formed by using several different processes. In various embodiments, the conductive base structure 40 is formed by using a metallic material to create a basic electrode shape with an overall length of 1000 μm (block 610).

Once the conductive base structure 40 is formed, a first set 48 of pores 52 is formed on at least a portion of the surface of the conductive base structure 40 (block 620). In some embodiments, the first pore set 48 is formed by creating pores 1/100^{th} the dimension of the overall length of the electrode 38 by using a laser ablation process, yielding an average pore dimension 56 of about 100 μm. In other embodiments, the processes that may be used to create the pores 52 of the first pore set 48 include, but are not limited to, electrical discharge machining (EDM) melt processing, sintering, plating, chemical etching, and depositing another material in a powdered or fragmented form.

After forming the first pore set 48, a second pore set 54 with a second but smaller pore dimension 64 is formed on at least a portion of the surface of the first pore set 48 (block 630). In some embodiments, the second pore set 54 is formed by creating pores 1/100^{th} the dimension 56 of the pores 52 of the first pore set 48 by using a second laser ablation process, yielding a second pore set 54 with an average pore dimension 64 of about 10 μm.

After forming the second pore set 54, a third pore set 58 with a smaller pore dimension 72 than that of second pore set 64 is formed on at least a portion of the surface of the second pore set 54 (block 640). In some embodiments, the third pore set 58 is formed by creating pores 1/100^{th} the dimension 64 of the pores 62 of the second pore set 54 by using a micro-abrasive blasting process with appropriately sized particles to yield an average pore dimension 72 of about one micron. Micro-abrasive blasting is a dry abrasive blasting process that delivers a stream of abrasives under high pressure via a small nozzle to a small area that ranges in size from about 1 mm² to about 3 cm². The abrasive media particle sizes can range from about 10 μm to about 150 μm.

After forming the third pore set 58, a fourth pore set 60 with a pore dimension 74 smaller than that of the third pore set 72 is formed on at least a portion of the surface of the third pore set 58 (block 650). In some embodiments, the fourth pore set 60 is formed with pores 1/100^{th} the dimension 72 of the pores 68 of the third pore set 58 by using a micro-abrasive blasting process with smaller sized particles to yield an average pore dimension 74 of about 100 nm (or 0.1 μm).

After forming the fourth pore set 60, a fifth pore set with a pore dimension smaller than that of the fourth pore set 74 is formed on at least a portion of the surface of the fourth pore set 60 (block 660). In some embodiments, the fifth pore set is formed with pores 1/100^{th} the dimension 74 of the pores 70 of the fourth pore set 60 by using a de-alloying process to yield an average pore dimension of about 10 nm (or 0.01 μm). De-alloying is a selective leaching process that removes a less noble metal component from a given material through a microscopic-scale galvanic corrosion mechanism. Ideal alloys are metals alloys made up of metal constituents with high distances in the galvanic series. Elements removed from this type of process may include, but are not limited to, zinc, aluminum, iron, cobalt, chromium, and carbon.

Alternatively, in other embodiments, the fifth pore set may also be formed and added /to the fourth pore set 60 using a deposition process. A deposition process adds material particles to a surface ranging in size from fractions of a nanometer to several micrometers. There are several forms of material deposition that may include, but not limited to, chemical deposition, physical vapor deposition, and reactive sputtering.

After forming the fifth layer, a sixth pore set with a pore dimension smaller than that of the fifth pore set is formed on at least a portion of the surface of the fifth pore set (block 670). In some embodiments, the sixth pore set is formed with pores 1/100^{th} the dimension of the pores of the fifth pore set by using a deposition process to yield an average pore dimension of about 1 nm (or 0.001 μm).

After forming the sixth layer, a seventh pore set with a pore dimension smaller than that of the sixth pore set is formed on at least a portion of the surface of the sixth pore set (block 680). In some embodiments, the seventh pore set is formed with pores 1/100^{th} the dimension of the pores of the sixth pore set by using a deposition process to yield an average pore dimension of about 0.1 nm (or 0.0001 μm).

All seven pore sets together create the porous structure 42 on the surface of the electrode 38 (block 690).

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as falling within the scope of the claims, together with all equivalents thereof

We claim:

1. An implantable medical lead for stimulating or sensing a target tissue, the implantable medical lead comprising:
a lead body including a proximal end portion and a distal end portion;
an electrical conductor electrically connected to the proximal end portion of the lead body; and
at least one electrode connected to the distal end portion of the lead body and connected to the electrical conductor, the electrode including:
a conductive base structure;
a first set of pores formed on an outer surface of the conductive base structure, the first set of pores having an average first pore dimension of between about ¼^{th} and about 1/100^{th} an electrode dimension; and
a second set of pores formed on at least a portion of the first set of pores, the second set of pores having an average second pore dimension of between about ¼^{th} and about 1/100^{th} the average first pore dimension.

2. The implantable medical lead of claim 1, wherein the conductive base structure is made of one of platinum and platinum alloy.

3. The implantable medical lead of claim 1, wherein the conductive base structure is made of one of a stainless steel, nitinol, nickel-cobalt alloy, titanium, gold, niobium, tantalum, ruthenium, palladium, and palladium alloy.

4. The implantable medical lead of claim 1, wherein the conductive base structure has a shape selected from one of a mushroom, helical, cylindrical, ribbon, and spherical shape.

5. The implantable medical lead of claim 1, wherein the electrode dimension is one of the length, width, diameter, and thickness of the electrode.

6. The implantable medical lead of claim 1, wherein the average first pore dimension is between about 10 and about 1000 μm.

7. The implantable medical lead of claim 1, further comprising the first set of pores having an average first pore dimension of between about 1/15^{th} and about 1/16^{th} an electrode dimension.

8. The implantable medical lead of claim 1, further comprising the second set of pores having an average second pore dimension of between about 1/15^{th} and about 1/16^{th} the average first pore dimension.

9. The implantable medical lead of claim 1, further comprising a third set of pores formed on at least a portion of the second set of pores, the third set of pores having an average third pore dimension of between about ¼^{th} and about 1/100^{th} the average second pore dimension.

10. The implantable medical lead of claim 1, further comprising the third set of pores having an average third pore dimension of between about 1/15^{th} and about 1/16^{th} the average second pore dimension.

11. The implantable medical lead of claim 1, further comprising a fourth set of pores formed on at least a portion of the third set of pores, the fourth set of pores having an average fourth pore dimension of between about ¼^{th} and about 1/100^{th} the average third pore dimension.

12. A method for manufacturing an electrode that is configured on an implantable medical lead body used for simulating or sensing a target tissue, the method comprising:
creating an electrode with a porous structure comprising:
forming a conductive base structure;
forming a first set of pores on at least a portion of the conductive base structure, the first set of pores having an average first pore dimension of between about ¼^{th} and about 1/100^{th} an electrode dimension; and
forming a second set of pores having a plurality of pores having an average second pore dimension of between about ¼^{th} and about 1/100^{th} the average first pore dimension.

13. The method of claim 12, wherein one or both of the forming steps are performed using a laser ablation process.

14. The method of claim 12, wherein one or both of the forming steps are performed using one of a chemical etching, micro-abrasive blasting, and an EDM melt process.

15. The method of claim 12, one or both of the forming steps are performed using one of a material deposition process and de-alloying process.

16. The method of claim 12, further comprising forming a third set of pores having a plurality of pores having an average third pore dimension of between about ¼^{th} and about 1/100^{th} the average second pore dimension.

17. The method of claim 16, further comprising forming a fourth set of pores having a plurality of pores having an average fourth pore dimension of between about ¼^{th} and about 1/100^{th} the average third pore dimension.

18. The method of claims 16, wherein the fourth set of pores is formed by a process that one of adds to, removes, displaces, and changes the material of the conductive base structure.

19. The method of claims 16, wherein the forming steps are performed using a laser ablation process.

20. The method of claims 16, wherein the forming steps are performed using one of a chemical etching, micro-abrasive blasting, and an EDM melt process.

21. The method of claims 16, wherein the forming steps are performed using one of a material deposition process and de-alloying process.
